(19) United States
(12) Patent Application Publication
(10) Pub. No.: US 2018/0009790 A1
(43) Pub. Date: Jan. 11, 2018

(54) AN IMPROVED PROCESS FOR THE PREPARATION OF HCV INHIBITOR

(71) Applicants: PRASAD RAJU VNKV VETUKURI, HYDERABAD (IN); GOVERDHAN GILLA, HYDERABAD (IN); RAJESH KUMAR RAPOLU, HYDERABAD (IN); KRISHNA PRASAD CHIGURUPATI, HYDERABAD (IN); GRANULES INDIA LIMITED, HYDERABAD (IN)

(72) Inventors: PRASAD RAJU VNKV VETUKURI, HYDERABAD (IN); GOVERDHAN GILLA, HYDERABAD (IN); RAJESH KUMAR RAPOLU, HYDERABAD (IN); KRISHNA PRASAD CHIGURUPATI, HYDERABAD (IN)

(21) Appl. No.: 15/532,515

(22) PCT Filed: Dec. 24, 2015
(86) PCT No.: PCT/IB2015/059982

§ 371 Date: Jun. 2, 2017

(30) Foreign Application Priority Data
Dec. 24, 2014 (IN) 6558/CHE/2014

(51) Int. Cl. C07D 403/14

(52) U.S. Cl. CPC C07D 403/14

(57) ABSTRACT

The present invention provides a novel process for the preparation of Ledipasvir of Formula I and its pharmaceutically acceptable salts.

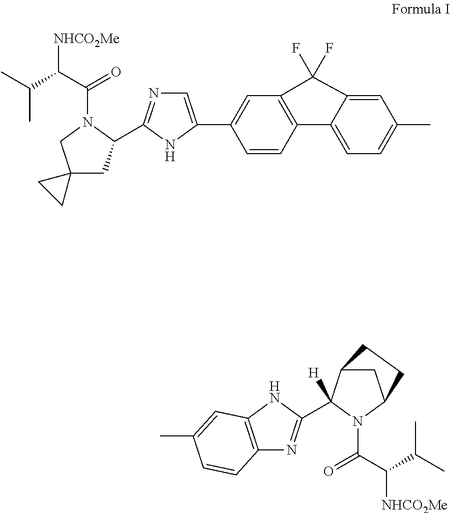

### FIELD OF INVENTION

The present invention provides a novel process for the preparation of Ledipasvir of Formula I and its pharmaceutically acceptable salts.

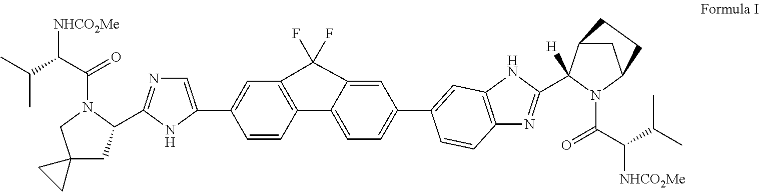

### BACKGROUND OF THE INVENTION

Ledipasvir is an inhibitor of the hepatitis C virus NS5A protein. Ledipasvir (formerly GS-5885) is a drug for the treatment of hepatitis C that was developed by Gilead Sciences. Ledipasvir/Sofosbuvir fixed-dose combination tablet for genotype 1 hepatitis C was approved recently by the USFDA with Harvoni Brand name. The ledipasvir/Sofosbuvir combination is a direct-acting antiviral agent that interferes with HCV replication and can be used to treat patients with genotypes 1a or 1b without PEG-interferon or ribavirin.

Harvoni is the first combination pill approved to treat chronic HCV genotype 1 infection. It is also the first approved regimen that does not require administration with interferon or ribavirin. Both drugs in Harvoni interfere with the enzymes needed by HCV to multiply. Sofosbuvir is a previously approved HCV drug marketed under the brand name Sovaldi.

Hepatitis C is a viral disease that causes inflammation of the liver that can lead to diminished liver function or liver failure. Most people infected with HCV have no symptoms of the disease until liver damage becomes apparent, which may take decades. Some people with chronic HCV infection develop scarring and poor liver function (cirrhosis) over many years, which can lead to complications such as bleeding, jaundice (yellowish eyes or skin), fluid accumulation in the abdomen, infections and liver cancer.

Ledipasvir inhibits an important viral phosphoprotein, NS5A, which is involved in viral replication, assembly, and secretion. Sofosbuvir, on the other hand, is metabolized to the active uridine analog triphosphate, which acts as a RNA chain terminator when incorporated into the RNA via the NS5B polymerase.

Ledipasvir chemically known as (1-{3-[6-(9,9-difuoro-7-{2-[5-(2-methoxycarbonylamino-3-methyl-butyryl)-5-aza-spiro[2.4]hept-6-yl]-3H-imidazol-4-yl}-9H-fluoren-2-yl)-1H-benzoimidazol-2-yl]-2-aza-bicyclo[2.2.1]heptane-2-carbonyl}-2-methyl-propyl)-carbamic acid methyl ester, is known to be an effective anti-HCV agent, as described in WO 2010/132601.

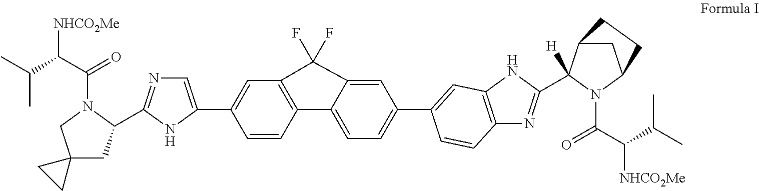

Though few synthetic methods have been reported in WO 2010/132601, it is desirable to discover new synthetic routes to Ledipasvir that can be executed on a large commercial scale resulting in industrially feasible process.

Also the synthesis of Ledipasvir reported in the literature involves costly, expensive, hazardous reagents which are difficult to handle at commercial scale. This problem needs to be addressed so that large scale manufacturing of Ledipasvir becomes commercially and economically viable.

There are number of cost-limiting raw materials and intermediates involved in the known methods of Ledipasvir synthesis which needs to be optimized in order to make Ledipasvir economically viable.

### OBJECTIVE OF THE INVENTION

The first embodiment of the present invention is to provide a novel process for the preparation of Ledipasvir of Formula I.

The second embodiment of the present invention is to provide an improved process for the preparation of various Salts, Solvates, Hydrates of Ledipasvir of Formula I.

The third embodiment of the present invention is to provide an improved process for the preparation of novel intermediates for the preparation of Ledipasvir of Formula I,

The fourth embodiment of the present invention is to provide an improved process for the preparation of Acid addition salts of intermediates of Ledipasvir of Formula I.

The fifth embodiment of the present invention is to provide an improved process for the purification of intermediates of Ledipasvir of Formula I.

The sixth embodiment of the present invention is to provide an improved process for the preparation of Novel Acid addition salts of Ledipasvir of Formula I.

The seventh embodiment of the present invention is to provide a one pot process without isolation of the intermediates for the preparation of Ledipasvir of Formula I.

### SUMMARY OF THE INVENTION

Accordingly, the present invention provides a process for the preparation of a Ledipasvir of Formula I or a pharmaceutically acceptable salt or solvate thereof

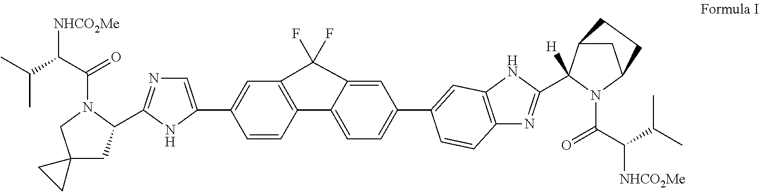

which comprises:
(i) reacting the compound of Formula VIa or its salts

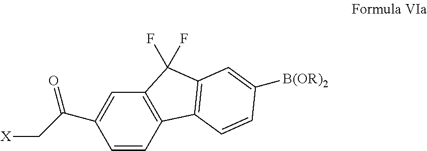

wherein X is halo or leaving group, R is C₁₋₆ alkyl, aryl or R groups combined together to form cycloalkyl group, with the compound of Formula V or its salts

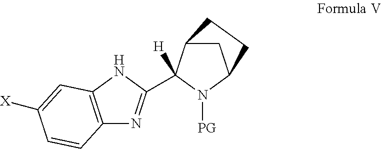

wherein X is as defined above, PG represents a protecting group or the group of Formula

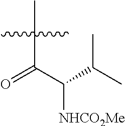

to give a compound of Formula VII,

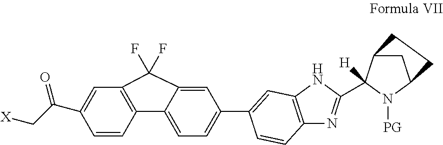

(ii) optional deprotecting the compound of Formula VII when PG represents a protecting group followed by reaction with compound of Formula

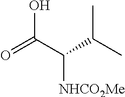

or its reactive derivative.
(iii) coupling the compound of Formula VII with compound of Formula VIh or its salts

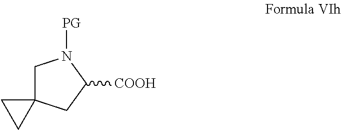

to give compound of Formula IX,

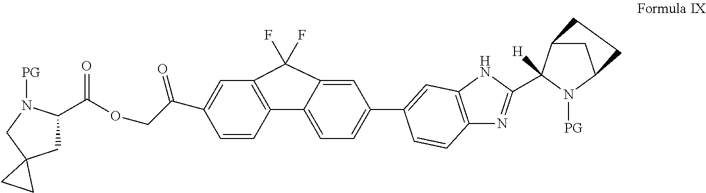

(iv) converting compound of formula IX to compound of formula IXi, and

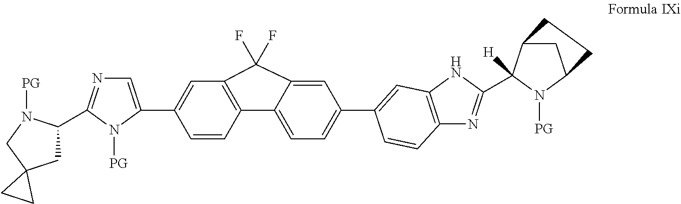

(v) optional deprotecting the compound of Formula IXi when PG represents a protecting group followed by reaction with compound of Formula

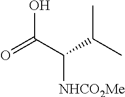

or its reactive derivative to give Ledipasvir or Formula I.

In one aspect, the present invention provides an improved process for the preparation of Ledipasvir compound of formula T or a pharmaceutically acceptable salt or solvate thereof, which comprises:
(i) cyclizing compound of Formula IX

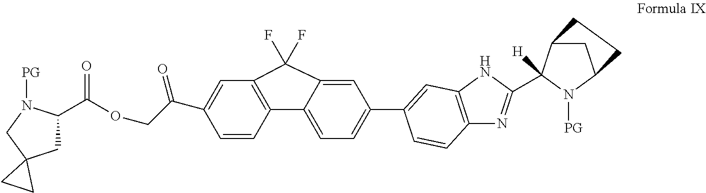

wherein PG represents protecting group or the group of Formula

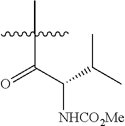

in the presence of a cyclizing agent and a solvent to give compound of formula IXi, and

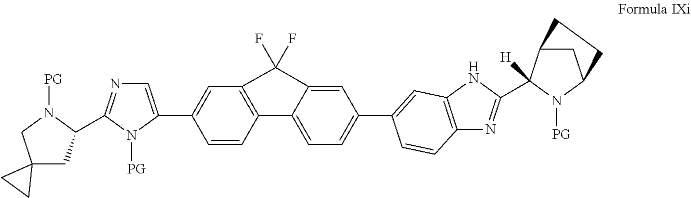

(ii) optional deprotecting the compound of Formula IXi when PG represents a protecting group followed by reaction with compound of Formula

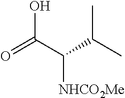

or its reactive derivative to give Ledipasvir of Formula I.

In another aspect, the present invention provides an improved process for the preparation of Ledipasvir compound of formula I or a pharmaceutically acceptable salt or solvate thereof, which comprises:
(i) reacting compound of Formula VII

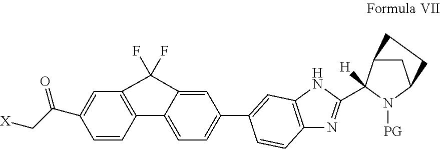

wherein X is halo or leaving group, PG represents N-protecting group or the group of Formula

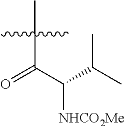

with compound of Formula VIh or its salts

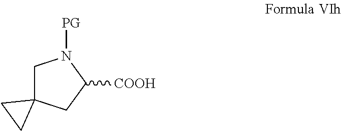

to give, compound of Formula IX,

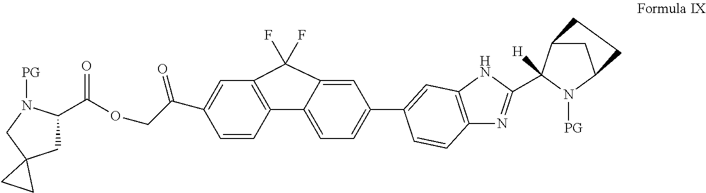

(ii) converting compound of formula IX to Ledipasvir of Formula I.

In another aspect, the present invention provides an improved process for the preparation of Ledipasvir compound of formula I or a pharmaceutically acceptable salt or solvate thereof, which comprises:
(i) reacting compound of Formula VII

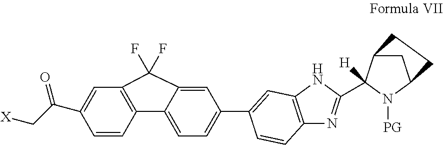

wherein X is halo or leaving group, PG represents N-protecting group or the group of Formula

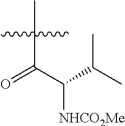

with compound of Formula VIh or its salts

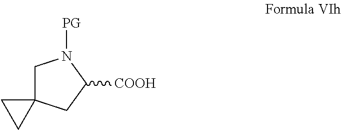

to give compound of Formula IX,

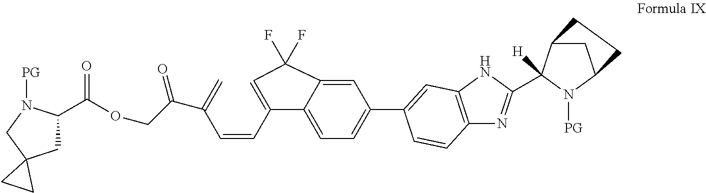

wherein PG represents N-protecting group or the group of Formula

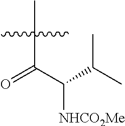

(ii) cyclizing compound of Formula IX in the presence of a cyclizing agent and a solvent to give compound of formula IXi, and

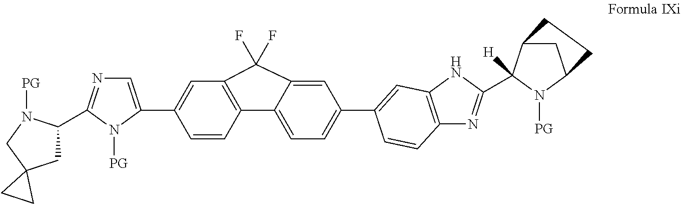

(iii) converting compound of formula IXi to Ledipasvir of Formula I.

In another aspect, the present invention provides compound of formula IX or its salts, an intermediate of Ledipasvir.

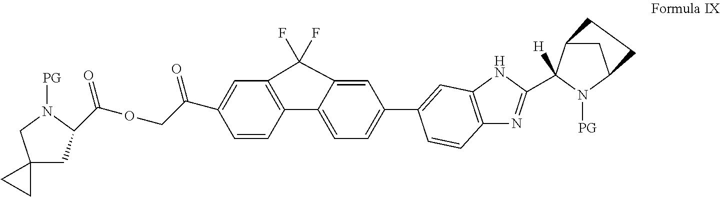

wherein PG represents protecting group

In another aspect, the present invention provides an improved process for the preparation of Ledipasvir of Formula I or a pharmaceutically acceptable salt or solvate thereof which compounds:
(i) coupling the compound of Formula V

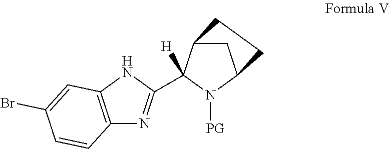

wherein PG is conventional protecting group, which can he deprotected using appropriate reagents, or a group of Formula

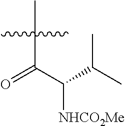

with compound of Formula VI

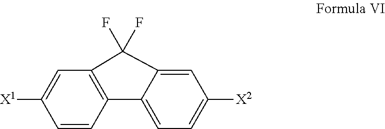

wherein X¹ represents halogen or a leaving group, and X² represents halogen or boronate esters by employing metal catalyst in an solvent to give compound of Formula VIIi

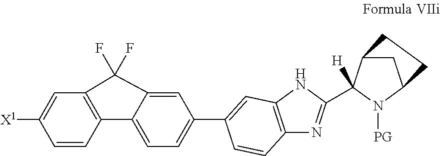

wherein PG, X¹ is as defined above,
(ii) condensation of compound of Formula VIIi with compound of Formula VIII

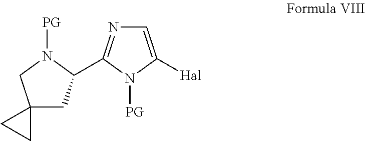

wherein PG is as defined above and Hal means halogen in a solvent to give compound of Formula IXi

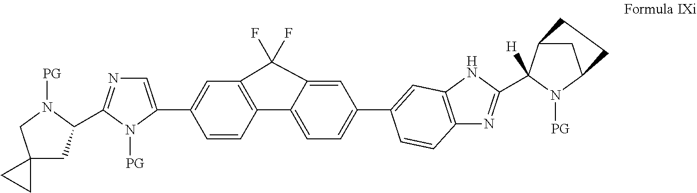

wherein PG is as defined above, and
(iii) deprotecting the compound of Formula IXi when PG represents a conventional protecting group followed by peptide coupling with 2-methoxycarbonylamino-3-methylbutyric acid of the Formula

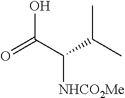

or its reactive derivative to give Ledipasvir of Formula I.

In yet another aspect, the present invention provides an alternative process for the preparation of the Ledipasvir of the Formula I
which comprises:
(i) reacting the compound of Formula VIa

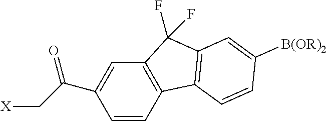

with the compound of Formula VIh

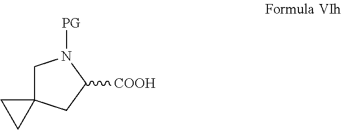

or its salts wherein PG represents a Protecting group or the group of Formula

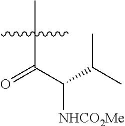

to give a compound of Formula VIi,

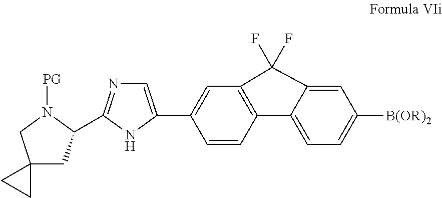

(ii) optional deprotecting the compound of Formula VIi when PG represents a protecting group followed by reaction with compound of Formula

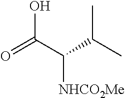

or its reactive derivative,
(iii) coupling the compound of Formula VIi with compound of Formula V

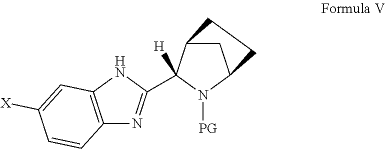

wherein X is halo or leaving group, to give compound of Formula IXi, and

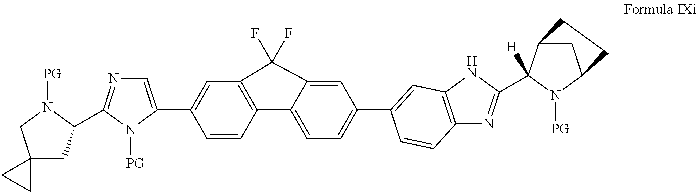

(iv) optional deprotecting the compound of Formula IXi when PG represents a protecting group followed by reaction with compound of Formula

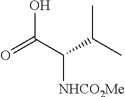

or its reactive derivative to give Ledipasvir of Formula I.

In yet another aspect, the present invention provides an improved process for the preparation of Ledipasvir compound of formula I or a pharmaceutically acceptable salt or solvate thereof, which comprises:
(i) reacting compound of Formula VIb

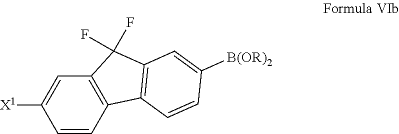

wherein X¹ is halo or leaving group, with the compound of Formula VIh or its salts

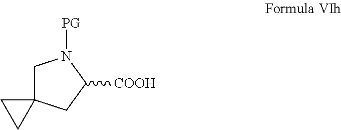

wherein PG represents a protecting group or the group of Formula

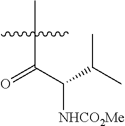

to give a compound of Formula

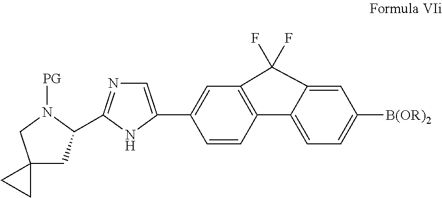

(ii) optional deprotecting the compound of Formula VIi when PG represents a protecting group followed by reaction with compound of Formula

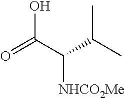

or its reactive derivative,
(iii) coupling the compound of Formula VIi with compound of Formula V

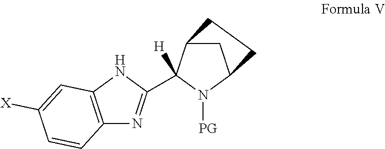

wherein X is halo or leaving group, to give compound of Formula IXi, and

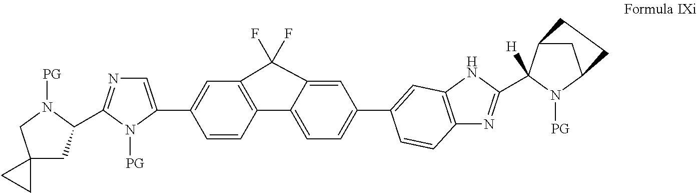

(iv) optional deprotecting the compound of Formula IXi when PG represents a protecting group followed by reaction with compound of Formula

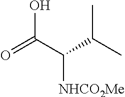

or its reactive derivative to give Ledipasvir of Formula I.

In another aspect, the present invention also provides a process for the preparation of compound of Formula VIa

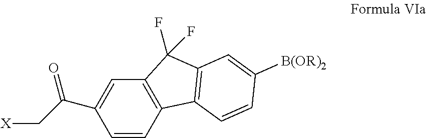

wherein X represents halogen or a leaving group; R represent hydrogen, an alkyl group, cycloalkyl group or other conventional boronate groups which comprises:
i) converting the compound of Formula VI

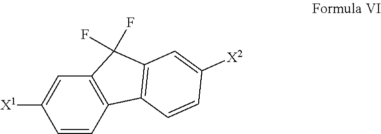

wherein X¹ and X² are halogens to give the compound of Formula VIc

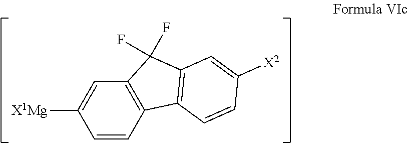

wherein X¹ and X² are as defined above
ii) coupling the compound of Formula VIc with compound of Formula VId

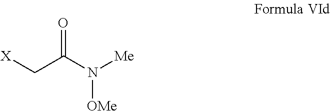

wherein X is halo or leaving group, to give compound of Formula VIe, and

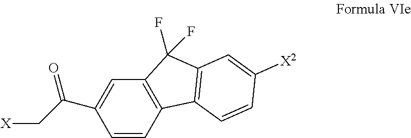

iii) converting the obtained compound of Formula VIe to compound of Formula VIa by reacting with boric acid or its derivatives such as trialkyl borates.

In yet another aspect, the present invention provides a process for the preparation of the compound of Formula VIb

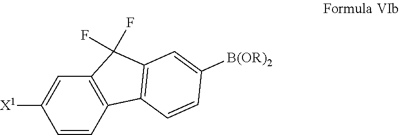

which comprises:
i) reacting the compound of Formula VI when X² represents halogen with organolithium reagents to give compound of Formula VIg having the following structure

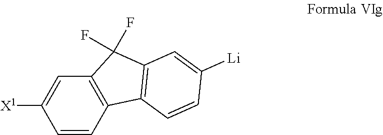

wherein X1 is as defined above, and
(ii) finally converting the compound of Formula VIg to compound of Formula VIb by reacting with boric acid or its derivatives such as trialkyl borates.

In yet another aspect, the compound of Formula VIb can also obtained by reacting the compound of Formula VI when X² represents halogen with boric acid or its derivatives such as trialkyl borates using a metal catalyst.

In yet another aspect, the present invention provides a process for the preparation of compound of Formula VIII

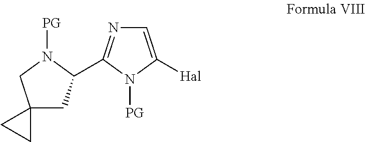

which comprises:
i) converting the compound of Formula C1 having the following structure

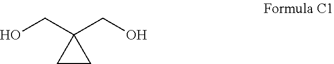

to compound of Formula C2 employing thionyl chloride in the presence of base to give compound of Formula C2

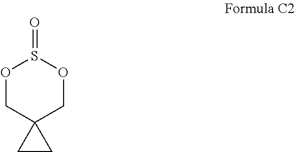

ii) halogenating the compound of Formula C2 to give compound of Formula C3

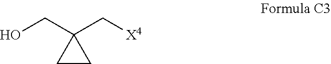

wherein X¹ represents halogen
iii) converting the compound of Formula C3 to give compound of Formula C4

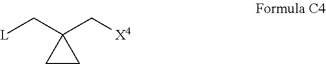

wherein L is a leaving group and X⁴ is as defined above,
iv) coupling the compound of Formula C4 with compound of Formula C5

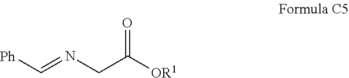

wherein R¹ represents hydrogen, alkyl or a chiral auxiliary, in the presence of a base to give compound of Formula C6

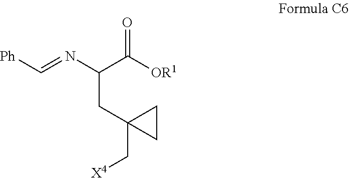

wherein R¹ and X⁴ are as defined above
v) converting the compound of Formula C6 to compound of Formula C7 under mild acidic conditions

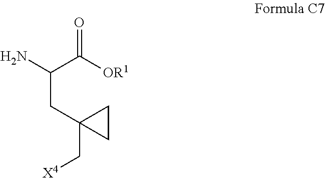

wherein R¹ and X⁴ arc as defined above
vi) the obtained compound of Formula C7 is cyclized to compound of Formula C8

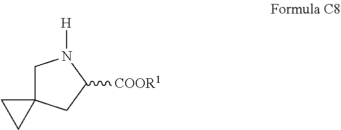

wherein R¹ is as defined above,
vii) optionally resolving the compound of Formula C8 when it is racemic and isolating the compound of Formula C8 as an acid addition salt of Formula C9

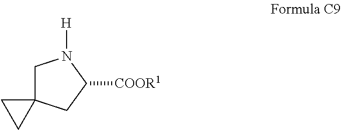

wherein R¹ represents hydrogen or alkyl group,
viii)protecting the compound of Formula C9 with a suitable protecting group to give compound of Formula C15 or its salts

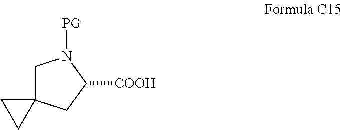

ix) converting the obtained protected compound of Formula C15 to compound of Formula C10

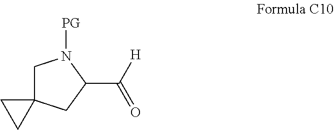

wherein PG represents protecting group
x) coupling the compound of Formula C10 with compound of Formula C11

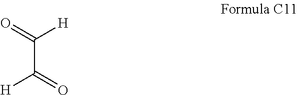

to give compound of Formula C12

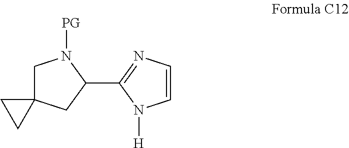

wherein PG represents protecting group, and
xi) converting the compound of Formula C12 to compound of Formula VIII.

In yet another aspect, the present inventions relates to an improved process for the preparation of compound of Formula VIII.

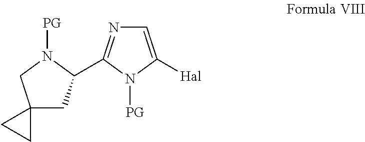

which comprises:
i) protecting the compound of Formula C8

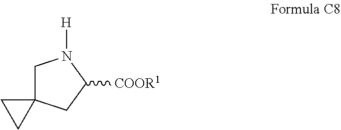

wherein R¹ is as defined above, to give compound of Formula C14

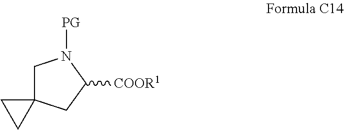

ii) optionally resolving the compound of Formula C14 when it is racemic and isolating the compound of Formula C15 or its salts

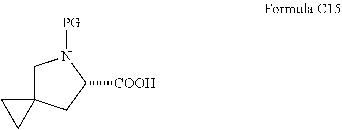

iii) converting the obtained protected compound of Formula C14 or Formula C15 to compound of Formula C10

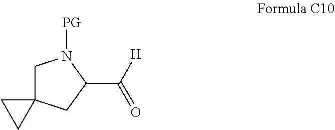

wherein PG represents protecting group
iv) coupling the compound of Formula C10 with compound of Formula C11

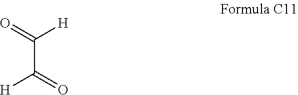

to give compound of Formula C12

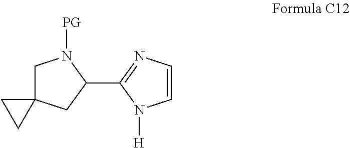

wherein PG represents protecting group, and
v) transforming the compound of Formula C12 to compound of Formula VIII.

In an another aspect, the present inventions provides a process for the resolution of compound of Formula C8 and Formula C14 which involves kinetic resolution or enzymatic hydrolysis of ester and hydrolysis of ester followed by resolution with chiral amine reagents.

### DETAILED DESCRIPTION OF THE INVENTION

As used in the present specification, the following words and phrases are generally intended to have the meanings as set forth below, except to the extent that the context in which they are used indicates otherwise.

Examples of suitable leaving groups X, X¹, X², X³ and L that can be applied in the process according to the invention are halogens, in particular Cl, Br, F or I; alkyl boronate esters, cycloalkyl boronate esters, mesyloxy, acyloxy, tosyloxy, benzyloxy, trifluoromethylsulfonyloxy, nonafluorobutylsulfonyloxy, (4-bromo-phenyl)sulfonyloxy, (4-nitro-phenyl)sulfonyloxy, (2-nitro-phenyl)sulfonyloxy, (4-isopropyl-phenyl)sulfonyloxy, (2,4,6-tri-isopropyl-phenyl)sulfonyloxy, (2,4,6-trimethyl-phenyl)sulfonyloxy, (4-rertbutyl-phenyl)sulfonyloxy, and (4-methoxy-phenyl)sulfonyloxyp. For practical reasons Cl is preferably chosen as leaving group.

The protecting group is selected from Carbobenzyloxy (Cbz), tert-Butyloxycarbonyl (BOC), p-Methoxybenzyl carbonyl (Moz or MeOZ), 9-Fluorenylmethyloxycarbonyl (FMOC), Acetyl (Ac), Benzoyl (Bz), Benzyl (Bn), benzyl Carbamate, p-Methoxybenzyl (PMB), 3,4-Dimethoxybenzyl (DMPM), p-methoxyphenyl (PMP), Tosyl (Ts), sulfonamides.

The compound of Formula VIa is coupled with compound of V employing metal catalyst in a solvent in the presence of a base yielding compound of Formula VII. This compound of Formula VII is condensed with compound of Formula VIh or its salts in the presence of solvent to give compound of Formula IX. The compound of formula IX is cyclized in the presence of suitable reagent in a solvent to give compound of formula IXi. The protecting groups on compound of Formula IXi are removed followed by peptide coupling with 2-methoxycarbonylamino-3-methyl-butyric acid in the presence of condensing agent to yield Ledipasvir in good yield.

Alternatively, the protecting groups of compound of Formula IXi are removed after the coupling with 2-methoxycarbonylamino-3-methyl butyric acid.

The compound of Formula V is coupled with compound of VI employing metal catalyst in a solvent yielding compound of Formula VIII. This compound of Formula VIIi is condensed with compound of Formula VIII to give compound of Formula IXi. The protecting groups on compound of Formula IXi are removed followed by peptide coupling with 2-methoxycarbonylamino-3-methyl-butyric acid to yield Ledipasvir in good yield.

Alternatively, the protecting groups of compound of Formula IXi are removed after the coupling with 2-methoxycarbonylamino-3-methyl butyric acid.

Accordingly the present invention provides a novel process for the preparation of Ledipasvir or its pharmaceutically acceptable salts. The compounds of formulae (C1-C15), (IV), (V), (VI), (VIa), (VIc), (VId), (VIe), (VIh), (VIi), (VIg), (VII), (VIIi), (VIII), (IX) and (IXi) or their salts used in the present invention may be isolated or not. Any of the above reactions may he carried out in-situ reactions to obtain Ledipasvir or its salts. The above compounds may isolated as salts or free bases, if the above compounds are isolated as salts they are converted to their free bases first and used for further reactions. Further, the above compound may isolated as crystalline Forms or isolated as an amorphous form or optionally recrystallized and used for further reactions.

“Solvent” as defined in the presence invention is selected from water or “alcohol solvents” such as methanol, ethanol, n-propanol, isopropanol, n-butanol and t-butanol and the like or “hydrocarbon solvents” such as benzene, toluene, xylene, heptane, hexane and cyclohexane and the like or “ketone solvents” such as acetone, ethyl methyl ketone, diethyl ketone, methyl tert-butyl ketone, isopropyl ketone and the like or “esters solvents” such as methyl acetate, ethyl acetate, propyl acetate, isopropyl acetate, n-butyl acetate, isobutyl acetate, sec-butyl acetate, and the like or “nitrile solvents” such as acetonitrile, propionitrile, butyronitrile and isobutyronitrile and the like or “ether solvents” such as di-tert-butylether, dimethylether, diethylether, diisopropyl ether, 1,4-dioxane, methyltert-butylether, ethyl tert-butyl ether, tetrahydrofuran, 2-methyl tetrahydrofuran, 2-methoxyethanol and dimethoxyethane, or “Amide solvents” such as formamide, DMF, DMAC, N-methyl-2-pyrrolidone, N-methylformamide, 2-pyrrolidone, 1-ethenyl-2-pyrrolidone and/or mixtures thereof.

“Base” as defined in the presence invention is selected from C₁₋₆ alkyl amines, NH₃, K₂CO₃, Na₂CO₃, NaHCO₃, NH₄OH, Mg(OH)₂, CaCO₃, Ca(OH)₂, KOH, NaOH, NaH, KH, KOtBu, CH₃COONa, CH₃COOK, (CH₃)₃CONa, LiOH, N-Methylmorpholine and/or mixtures thereof.

“Condensing agent” as defined in the presence invention is selected from HOBt, HBTU, TBTU, HOAt, DCC, EDC-HCl, CDI, BOP, T₃P and PyBOP or and/or mixtures thereof.

“Metal catalyst” as defined in the presence invention is selected from Palladium (0) or (II) complexes, selected from tetrakis(triphenylphosphine)palladium, tris(dibenzylideneacetone)dipalladium, palladium dppf chloride, Bis(triphenylphosphine)palladium(II) acetate, Bis(triethylphosphine)palladium(II) chloride.

“Cyclization” as defined in the presence invention is carried out in the presence of ammonium acetate in a solvent.

“Deprotection” as defined in the presence invention is carried out in the presence of metal catalyst, hydrogen source, wherein the metal catalyst is selected from Pd, Ni, Pt, Rh or the deprotection may carried out in the presence of an acid which is selected from strong acids such as HCl or CF₃COOH or the deprotection may carried out in the presence of a base, which is selected from primary or secondary amines.

“Boronate ester or its derivative” as defined in the presence invention is prepared using Boronate reagent which is selected from pinacolboronates, alkyl boronates and aryl boronates.

“Acid or acidic condition” as defined in the presence invention is selected from hydrochloric, hydrobromic, sulfuric, phosphoric, oxalic, maleic, succinic, citric, acetic and p-toluenesulfonic acid.

In a preferred embodiment, the present invention provides a process for the preparation of Ledipasvir of Formula I or a pharmaceutically acceptable salt or solvate thereof, which comprises:
(i) reacting the compound of Formula VIa or its salts

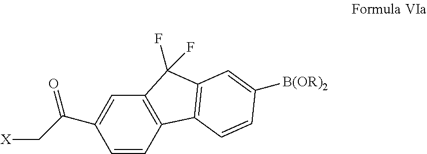

wherein X is halo or leaving group, R is C₁₋₆ alkyl, aryl or R groups combined together to form cycloalkyl group, with the compound of Formula V or its salts

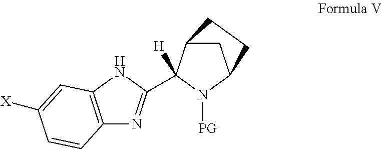

wherein X is halo or leaving group, wherein PG represents a protecting group or the group of Formula

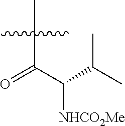

using a metal catalyst and a base in the presence of a solvent, to give a compound of Formula VII,

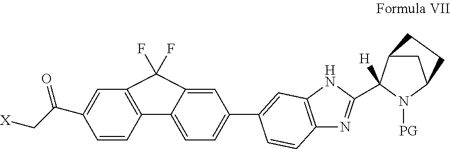

(ii) optional &protecting the compound of Formula VII when PG represents a protecting group in an acid reagent in a solvent followed by reaction with compound of Formula

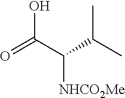

or its reactive derivative,
(iii) coupling the compound of Formula VII with compound of Formula VIh or its salts

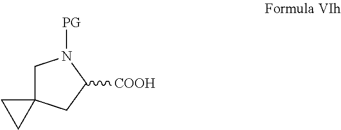

in the presence of a solvent and optionally using a catalyst to give compound of Formula IX,

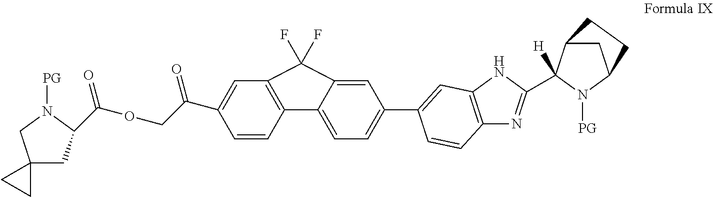

(iv) cyclizing compound of formula IX using a cyclizing agent in a solvent to give compound of formula IXi, and

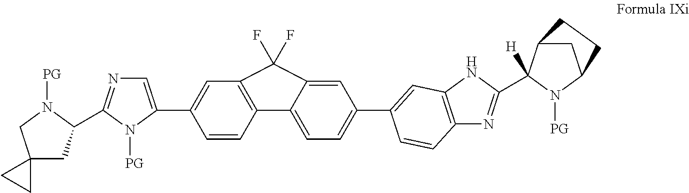

(v) optional deprotecting the compound of Formula IXi when PG represents a protecting group followed by reaction with compound of Formula

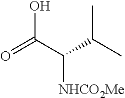

or its reactive derivative in the presence of a condensing agent and a solvent to give Ledipasvir of Formula I.

In a more preferred embodiment, the present invention provides a process for the preparation of a Ledipasvir of Formula I or a pharmaceutically acceptable salt or solvate thereof, which comprises:
(i) reacting the compound of Formula VIa or its salts

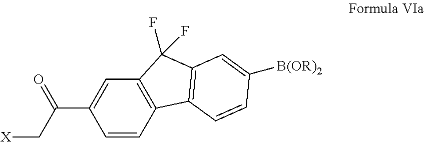

wherein X is Cl, R is a cycloalkyl group, with the compound of Formula V or its salts

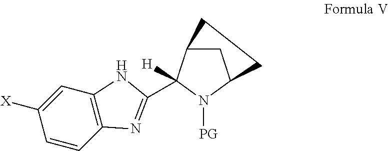

wherein PG represents a protecting group or the group of Formula

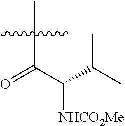

using palladium dppf chloride, palladium tetrakis triphenylphosphine and potassium carbonate in dimethyl ether and water to give a compound of Formula VII,

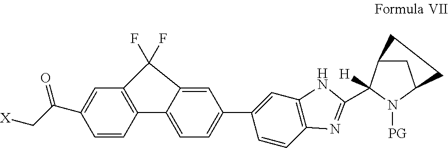

(ii) coupling the compound of Formula VII with compound of Formula VIh or its salts

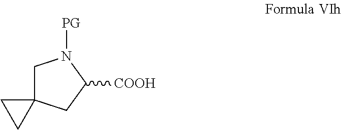

in acetone to give compound of Formula IX,

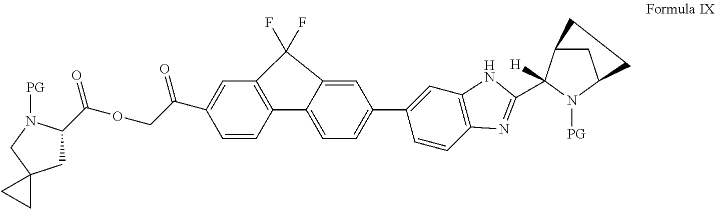

(iii) cyclizing compound of formula IX using ammonium acetate, in the presence of toluene and catalytic amount of 2-methoxy ethanol to give compound of formula IXi, and

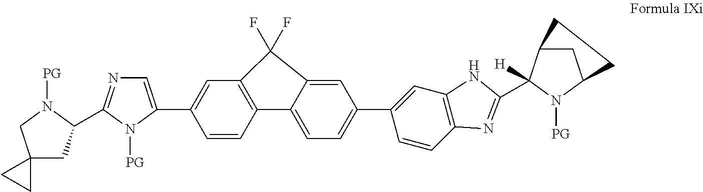

(iv) deprotecting the compound of Formula. IXi when PG represents a protecting group using HCl in acetonitrile followed by reaction with compound of Formula

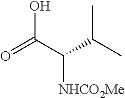

or its reactive derivative in the presence of dimethyl formamide, EDC.HCl, HOBt.H₂O and N-methyl morpholine to give Ledipasvir of Formula I.

In a preferred embodiment, the present invention provides an improved process for the preparation of Ledipasvir compound of formula I or a pharmaceutically acceptable salt or solvate thereof, which comprises:
(i) cyclizing compound of Formula IX

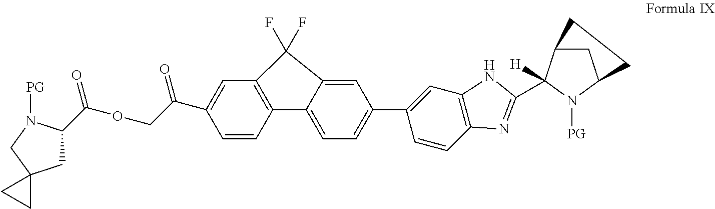

wherein PG represents protecting group or the group of Formula

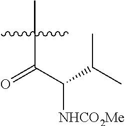

using ammonium acetate, in the presence of toluene and catalytic amount of 2-methoxy ethanol to give compound of formula IXi, and

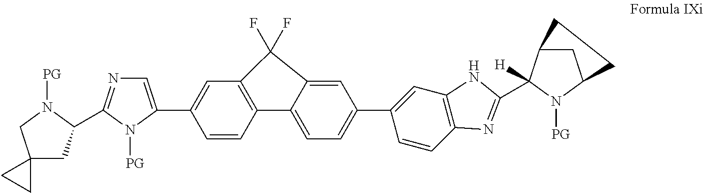

(ii) deprotecting the compound of Formula IXi when PG represents a protecting group using HCl in acetonitrile followed by reaction with compound of Formula

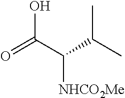

or its reactive derivative in the presence of dimethyl formamide, EDC.HCl, HOBt.H₂O and N-methyl morpholine to give Ledipasvir of Formula I.

In a preferred embodiment, the present invention provides an improved process for the preparation of Ledipasvir compound of formula I or a pharmaceutically acceptably suit or solvate thereof, which comprises:
(i) reacting compound of Formula VII

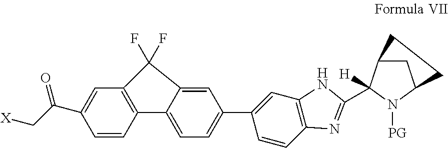

with compound of Formula VIh or its salts

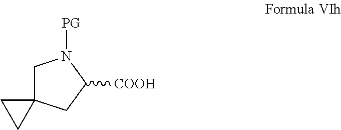

in acetone to give compound of Formula IX,

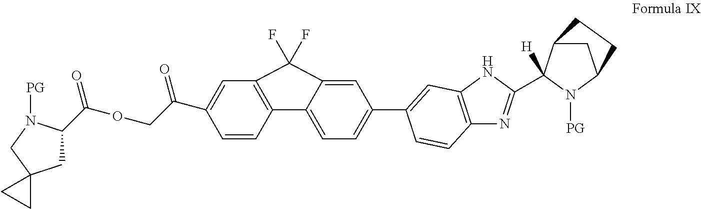

(ii) cyclizing compound of formula IX using ammonium acetate, in the presence of toluene and catalytic amount of 2-methoxy ethanol to give compound of formula IXi, and

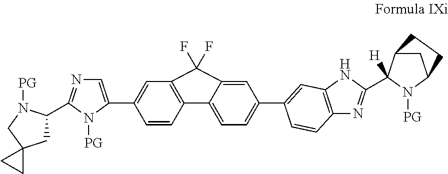

(iii) deprotecting the compound of Formula IXi when PG represents a protecting group using HCl in acetonitrile followed by reaction with compound of Formula

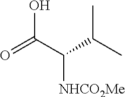

or its reactive derivative in the presence of dimethyl formamide, EDC.HCl, HOBt.H₂O and N-methyl morpholine to give Ledipasvir of Formula I.

The present invention also describes and improved process for the preparation of the Intermediate compounds of Formula VIII.

The compounds of Formula I and intermediates of Formula VIII are purified with solvents.

The compound of Formula VIa is an important coupling precursor for the preparation of Ledipasvir. The compound of Formula VIa′ is prepared by First converting the compound VI′ to VIc′ wherein the end product may be or may not be isolated. The compound of Formula VIc′ is then coupled with compound of Formula VId' to yield compound of Formula VIa′.

The preparation of compound of Formula VIa′ is depicted as below:

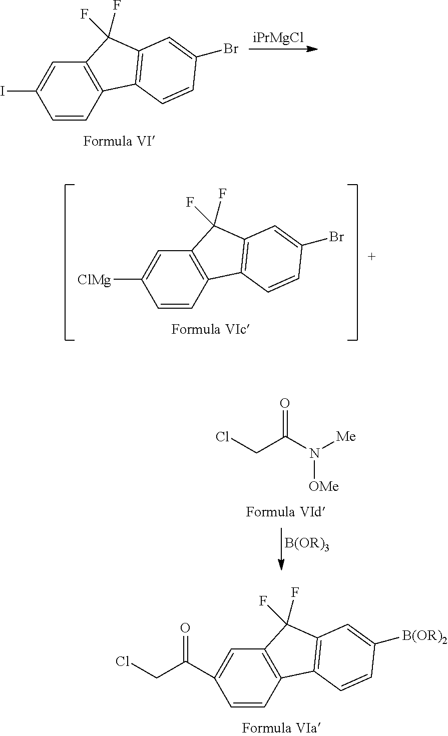

Alternatively, the compound of Formula VI′ is converted to the lithium derivative by treatment with organolithium reagents and the resulting compound of Formula VIg′ is converted to the boronate derivative of compound of Formula VIb′. The process is shown in the scheme given below:

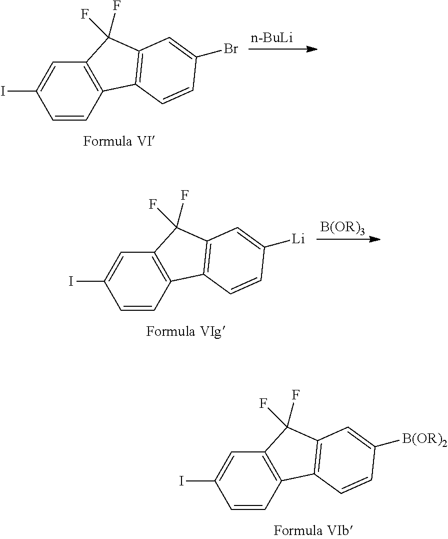

The compound of Formula VIII is an important intermediate during the synthesis of compound of Formula I. An improved methodology has been designed so as to enable the compound of Formula VIII in good yields and good purity. In this synthesis the compound of Formula C1 is first treated with thionyl chloride in the presence of base to give compound of Formula C2. The compound of Formula C2 is then converted to Compound of Formula C3 which is then converted in the presence of base like triethylamine to give the compound of Formula C4. This C4 compound is coupled with compound of Formula C5 in the presence of base to afford the compound of Formula C6 which is not isolated and taken up further under mild acidic conditions to give compound of Formula C7. This compound of Formula C7 is cyclized to compound of Formula C8 which is then hydrolysed under basic condition to give the hydroxyl compound which is then resolved under dynamic resolution method to afford compound of Formula C9. The compound of Formula C9 is optionally isolated as its acid addition salt. The compound of Formula C9 is converted to compound of Formula C10 followed by condensation to compound of Formula C11 to yield compound of Formula C12. The compound of Formula C12 is then transformed to compound of Formula VIII.

### EXAMPLE 1

### Preparation of 2-chloro-1-(9,9-difluoro-7-(4,4,5,5-tetramethyl-1,3,2-dioxaborolan-2-yl)-9H-fluoren-2-yl)ethanone

To (8 volumes) of tetrahydrofuran (THF), (0.2457 mol) of 2-bromo-9,9-difluoro-7-iodo-9H-fluorene was charged and the resultant reaction mixture was cooled to about −15° C. To this reaction mixture isopropyl magnesium chloride (1M in tetrahydrofuran (THF)) was added at about −15° C. and stirred for about 30 min. Then a solution of 2-chloro N,N-methylmethoxyacetamide in toluene was added at about −15° C. and stirred for about 90 min. Then the temperature of the resultant reaction mixture was raised to about 0° C. and stirred for about 30 min. Then 1N HCl was added to the reaction mixture and extracted with ethyl acetate for thrice. The organic layer was separated and dried with anhydrous sodium sulphate, the organic layer was separated and distilled under vacuum below 45° C. followed by isolation in isopropyl alcohol. To (20 volumes) of 1,4-dioxane the isolated solid was charged and stirred for about 15 min. Then added bis pinacolato diboron, potassium acetate and palladium dppf chloride and the temperature of the resultant reaction mixture was raised to about 90° C. and maintained at about 90-95° C. for about 16 hrs. Then cooled to about room temperature and diluted with water followed by extraction with ethyl acetate (3 times). The organic layer was separated and washed with brine solution, the organic layer was separated and dried with anhydrous sodium sulphate and then distilled the solvent completely at below 45° C. under vacuum to yield the title compound. Yield: 58%.

### EXAMPLE 2

### Preparation of (1R,3S,4S)-tert-butyl 3-(6-(7-(2-chloroacetyl)-9,9-difluoro-9H-fluoren-2-yl)-1H-benzo[d]imidazol-2-yl)-2-azabicyclo[2.2.1]heptane-2-carboxylate

To a mixture solution of dimethyl ether (DME) (7 volumes) and water (3 volumes) was charged 2-chloro-1-(9,9-difluoro-7-(4,4,5,5-tetramethyl-1,3,2-dioxaborolan-2-yl)-9H-fluoren-2-yl)ethanone and potassium carbonate. The resultant reaction mixture was stirred at room temperature for about 10 min., and was added palladium dppf chloride, palladium tetrakis triphenylphosphine, (1R,3S,4S)-tert-butyl-3-(6-bromo-1H-benzo[d]imidazol-2-yl)-2-azabicyclo-[2.2.1]heptane-2-carboxylate. The temperature of the resultant reaction mixture was raised to about 90° C. and stirred at 90-95° C. for about 16-18 hrs. Then cooled to room temperature and diluted with water followed by extraction with ethyl acetate (3 times). The organic layer was separated and washed with brine solution, then dried with anhydrous sodium sulphate and distilled the solvent completely at below 45° C. under vacuum to yield the title compound. Yield: 96%.

### EXAMPLE 3

### Preparation of 1,1-bis(iodomethyl)cyclopropane

To (5 volumes) of dichloro methane was charged a solution of imidazole and triphenylphosphine. The resultant reaction mixture was cooled to about 0° C. Then added a solution of iodine in dichloro methane (5 volumes) at about 0° C. for about 60 min. Then added a solution of cyclopropane-1,1-diyldimethanol in dichloro methane (5 volumes) at about 0° C. for about 30 min. and stirred at 10-15° C. for about 3 hrs. Then the reaction mass was diluted with brine solution at 10-15° C. The organic and aqueous layers were separated and to the organic layer n-heptane (10 volumes) was charged. The total organic layer was washed with saturated sodium sulphite solution (2 times). 70% of the organic layer was distilled at below 45° C. under vacuum. Then (10 volumes) of n-heptane was added and 12 volumes of the solvent was distilled at below 45° C. under vacuum. The slurry was filtered on silica bed and washed with n-heptane, the filterate mls were distilled below 45° C. under vacuum to yield the title compound. Yield: 46%

### EXAMPLE 4

### Preparation of 5-tert-butyl 6-ethyl 5-azaspiro[2.4]heptane-5,6-dicarboxylate

To dimethyl acetamide (4 volumes) was charged sodium hydride 60% dispersion in mineral oil and cooled to about 0° C. Then added 1,1-bis(iodomethyl)cyclopropane and solution of ethyl 2-(tert-butoxycarbonylamino)acetate in dimethyl acetamide (4 volumes) at 0-10° C. in 3 hrs. The resultant reaction mixture was stirred at 0-10° C. for about 2 hrs and then added acetic acid at the same temperature over about 3 hrs. The resultant reaction mixture was stirred at 0-10° C. for about 12 hrs. The reaction mass was diluted with (15 volumes) of methyl tertiary butyl ether (MTBE), and water (7 volumes). The organic and aqueous layers were separated, the organic layer was washed with saturated sodium bicarbonate solution and then with brine solution. The organic layer was distilled completely at below 45° C. under vacuum and then charged acetonitrile (3 volumes), n-hexane (2 volumes) to the oily mass. The obtained layers were separated and distilled at below 45° C. under vacuum to yield the title compound. Yield: 86%.

### EXAMPLE 5

### Preparation of potassium 5-(tert-butoxycarbonyl)-5-azaspiro[2.4]heptane-6-carboxylate

To water (1.5 volumes) was charged lithium hydroxide monohydrate, 2-methyl tetrahydrofuran (3.5 volumes). Then charged a solution of 5-tert-butyl 6-ethyl 5-azaspiro[2.4]heptane-5,6-dicarboxylate in 2-methyltetrahydrofuran (1.5 volumes) at room temperature. The temperature of the resultant reaction mixture was raised to 50-55° C. and maintained for about 24 hrs. The organic, aqueous layers were separated and the aqueous layer was diluted with 2-methyl-tetrahydrofuran (5 volumes) and added HCl (0.75 volumes). Then the organic, aqueous layers were separated and the organic layer was distilled at below 45° C. under vacuum. The obtained crude was diluted with 2-methyl tetrahydrofuran (5.5 volumes) and heated to about 40° C. and added potassium tert-butoxide solution (1 M in tetrahydrofuran (THF)) at same temperature and stirred for at 40° C. for about 1 hr. and then cooled to 10-15° C., stirred for 2 hrs. Further cooled to 5-10° C. and stirred for about 30 min. The precipitated solid was filtered and the solid obtained was washed with 2-methyltetrahydrofuran to yield the title compound. Yield: 60%.

### EXAMPLE 6

### Preparation of (S)-6-(2-(7-(2-((1R,3S,4S)-2-(tert-butoxycarbonyl)-2-azabicyclo[2.2.1]heptan-3-yl)-1H-benzo[d]imidazol-6-yl)-9,9-difluoro-9H-fluoren-2-yl)-2-oxoethyl) 5-tert-butyl 5-azaspiro[2.4]heptane-5,6-dicarboxylate

To acetone (10 volumes) was charged (1R,3S,4S)-tert-butyl 3-(6-(7-(2-chloroacetyl)-9,9-difluoro-9H-fluoren-2-yl)-1H-benzo[d]imidazol-2-yl)-2-azabicyclo[2.2.1]heptane-2-carboxylate and potassium 5-(tert-butoxycarbonyl)-5-azaspiro[2.4]heptane-6-carboxylate. The resultant reaction mixture was heated to about 55° C. and stirred at 50-55° C. for 5-6 hrs. Then added water (3 volumes) to the reaction mass, stirred for about 30 min. and again added water (1 volume), stirred at 40-45° C. for about 1 hr. Then cooled to room temperature and stirred for about 2 hrs. The precipitated solid was filtered and the solid obtained was washed with mixture of acetone and water to yield the title compound. Yield: 80%.

### EXAMPLE 7

### Preparation of (1R,3S,4S)-tert-butyl 3-(6-(7-(2-((S)-5-(tert-butoxycarbonyl)-5-azaspiro[2.4]heptan-6-yl)-1H-imidazol-5-yl)-9,9-difluoro-9H-fluoren-2-yl)-1H-benzo[d]imidazol-2-yl)-2-azabicyclo[2.2.1]heptane-2-carboxylate

To toluene (10 volumes) was added (S)-6-(2-(7-(2-((1R,3S,4S)-2-(tert-butoxycarbonyl)-2-azabicyclo[2.2.1]heptan-3-yl)-1H-benzo[d]imidazol-6-yl)-9,9-difluoro-9H-fluoren-2-yl)-2-oxoethyl) -tert-butyl 5-azaspiro[2.4]heptane-5,6-dicarboxylate, ammonium acetate and catalytic amount of 2-methoxyethanol. The resultant reaction mixture was heated to 90-95° C. and maintained for 5-6 hrs. n-heptane (10 volumes) was added to the reaction mass at 50-55° C. and stirred for about 1 hr. at the same temperature. Then cooled to room temperature and diluted with n-heptane (8 volumes) and stirred for about 2 hrs. The precipitated solid was filtered and washed with n-heptane to yield the title compound. Yield: 81%.

### EXAMPLE 8

### Preparation of 6-(7-(2-((S)-5-azaspiro[2.4]heptan-6-yl)-1H-imidazol-5-yl)-9,9-difluoro-9H-fluoren-2-yl)-2-((1R,3S,4S)-2-azabicyclo[2.2.1]heptan-3-yl)-1H-benzo[d]imidazole hydrochloride

To acetonitrile (5 volumes) was added (1R,3S,4S)-tert-butyl 3-(6-(7-(2-((S)-5-(tert-butoxycarbonyl)-5-azaspiro[2.4]heptan-6-yl)-1H-imidazol-5-yl)-9,9-difluoro-9H-fluoren-2-yl)-1H-benzo[d]imidazol-2-yl)-2-azabicyclo[2.2.1]heptane-2-carboxylate and heated to 60-65° C. 5N HCl was added to the reaction mass slowly at 60-65° C. and stirred for about 12 hrs. The reaction mass was diluted with acetonitrile (7 volumes) and allowed to room temperature. Then added acetonitrile (16 volumes) and stirred for about 2 hrs. at room temperature. The solid obtained was filtered and washed with acetonitrile to yield the title compound. Yield: 79%.

### EXAMPLE 9

### Preparation of methyl [(2S)-1-{(6S)-6-[5-(9,9-difluoro-7-{2-[(1R,3S,4S)-2-{(2S)-2-[(methoxycarbonyl)amino]-3-methylbutanoyl}-2-azabicyclo[2.2.1]hept-3-yl]-1H-benzimidazol-6-yl}-9H-fluoren-2-yl)-1H-imidazol-2-yl]-5-azaspiro[2.4]hept-5-yl}-3-methyl-1-oxobutan-2-yl]carbamate (Ledipasvir)

To dimethyl formamide (DMF) (8 volumes) was charged 6-(7-(2-((S)-5-azaspiro[2.4]heptan-6-yl)-1H-imidazol-5-yl)-9,9-difluoro-9H-fluoren-2-yl)-2-((1R,3S,4S)-2-azabicyclo[2.2.1]heptan-3-yl)-1H-benzo[d]imidazole hydrochloride, ethylene dichloride. HCl, HOBt.H₂O and MOC-L-valine at room temperature. N-methyl morpholine was added at 0-5° C. Temperature of the resultant reaction mixture was raised to room temperature and maintained for about 16 hrs. The reaction mass was diluted with water (15 volumes) and extracted with ethyl acetate (3 times). The organic layer was washed with water (4 times), brine (2 times) and the organic layer was dried with anhydrous sodium sulphate and distilled at below 45° C. under vacuum and isolated the solid in acetone to afford the pure title compound. Yield: 49%.

1. (canceled)

2. A process for the preparation of Ledipasvir of Formula I or a pharmaceutically acceptable salt or solvate thereof, which comprises:
(i) reacting the compound of Formula VIa or its salts 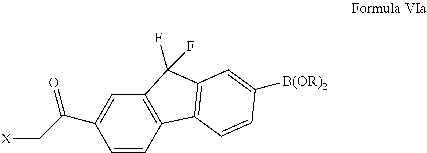
wherein X is halo or leaving group, R is C₁₋₆ alkyl, aryl or R groups combined together to form cycloalkyl group, with the compound of Formula V or its salts 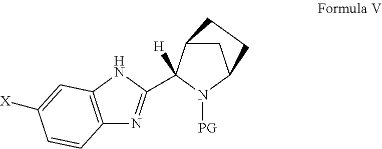
wherein X is as defined above, PG represents a protecting group or the group of Formula 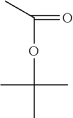
using a metal catalyst and a base in the presence of a solvent, to give a compound of Formula VII, 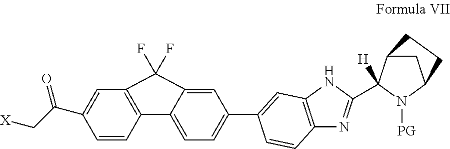
(ii) optional deprotecting the compound of Formula VII when PG represents a protecting group in an acid reagent in a solvent followed by reaction with compound of Formula 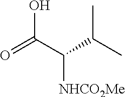
or its reactive derivative,
(iii) coupling the compound of Formula VII with compound of Formula VIh or its salts 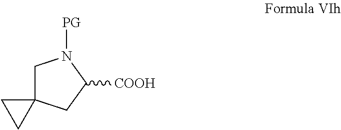
in the presence of a solvent and optionally using a catalyst to give compound of Formula IX, 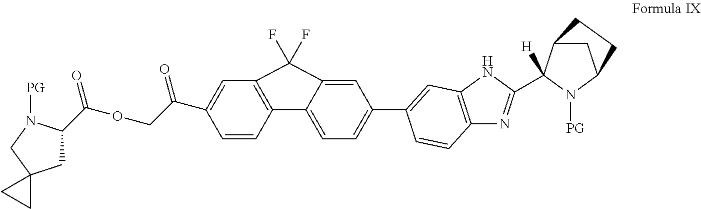
(iv) cyclizing compound of formula IX using a ammonium acetate in a solvent to give compound of formula IXi, and 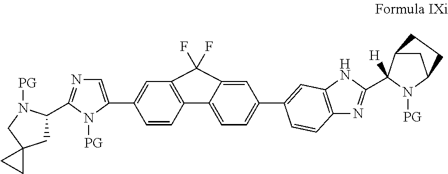
(iv) optional deprotecting the compound of Formula IXi when PG represents a protecting group in an acid reagent in a solvent followed by reaction with compound of Formula 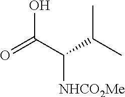
or its reactive derivative in the presence of a condensing agent and a solvent to give Ledipasvir of Formula I.

3. The process for the preparation of Ledipasvir of Formula I or a pharmaceutically acceptable salt or solvate thereof according to claim 2, wherein the metal catalyst used in step (i) is selected from Palladium (0) or (II) complexes, selected from tetrakis(triphenylphosphine)palladium, tris(dibenzylideneacetone)dipalladium, palladium dppf chloride, Bis(triphenylphosphine)palladium(II) acetate, Bis(triethylphosphine)palladium(II) chloride and/or mixtures thereof.

4. The process for the preparation of Ledipasvir of Formula I or a pharmaceutically acceptable salt or solvate thereof according claim 2, wherein the base used in step (i) is selected from K₂CO₃, Na₂CO₃, NaHCO₃, Mg(OH)₂, CaCO₃, Ca(OH)₂, KOH, NaOH, KOtBu, LiOH, and acid reagent used in steps (ii) and (v) is selected from hydrochloric, hydrobromic, sulfuric, phosphoric, oxalic, maleic, succinic, citric, acetic and p-toluenesulfonic acid and/or mixtures thereof.

5. (canceled)

6. The process for the preparation of Ledipasvir of Formula I or a pharmaceutically acceptable salt or solvate thereof according to claim 2, wherein the catalyst used in step (iii) is selected from acid or base, wherein the acid is selected from hydrochloric, hydrobromic, sulfuric, phosphoric, oxalic, maleic, succinic, citric, acetic and p-toluenesulfonic acid and/or mixtures thereof and base is selected from C₁₋₆ alkyl amines, NH₃, K₂CO₃, Na₂CO₃, NaHCO₃, NH₄OH, Mg(OH)₂, CaCO₃, Ca(OH)₂, KOH, NaOH, NaH, KH, KOtBu, CH₃COONa, CH₃COOK, (CH₃)₃CONa, LiOH, N-Methylmorpholine and/or mixtures thereof.

7. (canceled)

8. The process for the preparation of Ledipasvir of Formula I or a pharmaceutically acceptable salt or solvate thereof according to claim 2, wherein the condensing agent used in step (v) is selected from HOBt, HBTU, TBTU, HOAt, DCC, CDI, EDC-HCl, BOP, T₃P and PyBOP or and/or mixtures thereof.

9. The process for the preparation of Ledipasvir of Formula I or a pharmaceutically acceptable salt or solvate thereof according to claim 2, wherein the solvent used is water or “alcohol solvents” such as methanol, ethanol, n-propanol, isopropanol, n-butanol and t-butanol and the like or “hydrocarbon solvents” such as benzene, toluene, xylene, heptane, hexane and cyclohexane and the like or “ketone solvents” such as acetone, ethyl methyl ketone, diethyl ketone, methyl tert-butyl ketone, isopropyl ketone and the like or “esters solvents” such as methyl acetate, ethyl acetate, propyl acetate, isopropyl acetate, n-butyl acetate, isobutyl acetate, sec-butyl acetate, and the like or “nitrile solvents” such as acetonitrile, propionitrile, butyronitrile and isobutyronitrile and the like or “ether solvents” such as di-tert-butylether, dimethylether, diethylether, diisopropyl ether, 1,4-dioxane, methyltert-butylether, ethyl tert-butyl ether, tetrahydrofuran, 2-methyl tetrahydrofuran, 2-methoxyethanol and dimethoxyethane, or “Amide solvents” such as formamide, DMF, DMAC, N-methyl-2-pyrrolidone, N-methylformamide, 2-pyrrolidone, 1-ethenyl-2-pyrrolidone and/or mixtures thereof.

10. The process for the preparation of Ledipasvir of Formula I or a pharmaceutically acceptable salt or solvate thereof according to claim 2, which comprises:
(i) reacting the compound of Formula VIa or its salts 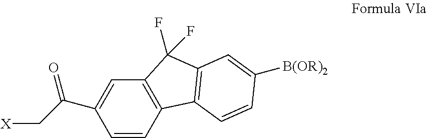
wherein X is Cl, R is a cycloalkyl group, with the compound of Formula V or its salts 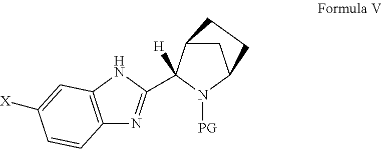
wherein PG represents a protecting group or the group of Formula 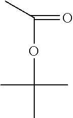
using palladium dppf chloride, palladium tetrakis triphenylphosphine and potassium carbonate in dimethyl ether and water to give a compound of Formula VII, 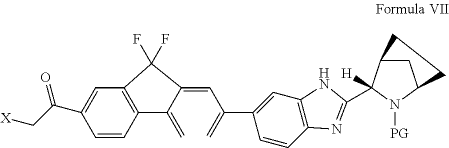
(ii) coupling the compound of Formula VII with compound of Formula VIh or its salts 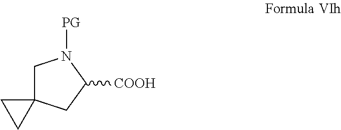
in acetone to give compound of Formula IX, 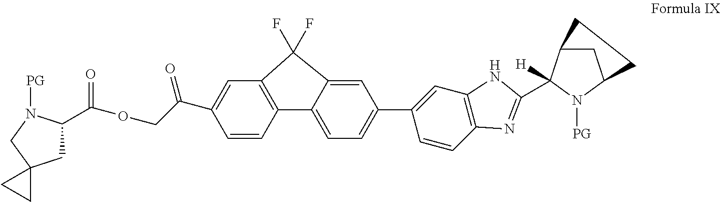
(iii) cyclizing compound of formula IX using ammonium acetate, in the presence of toluene and catalytic amount of 2-methoxy ethanol to give compound of formula IXi, and 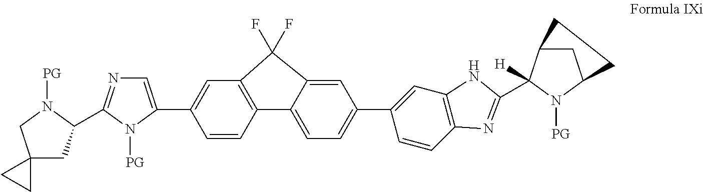
(iv) deprotecting the compound of Formula IXi when PG represents a protecting group using HCl in acetonitrile followed by reaction with compound of Formula 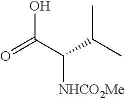
or its reactive derivative in the presence of dimethyl formamide, EDC.HCl, HOBt.H₂O and N-methyl morpholine to give Ledipasvir of Formula I.

11. An improved process for the preparation of Ledipasvir compound of formula I or a pharmaceutically acceptable salt or solvate thereof, which comprises:
(i) cyclizing compound of Formula IX 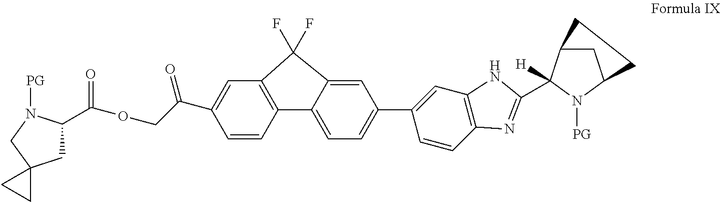
wherein PG represents protecting group or the group of Formula 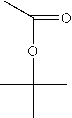
in the presence of a ammonium acetate and in toluene and a catalytic amount of 2-methoxy ethanol to give compound of formula IXi, and 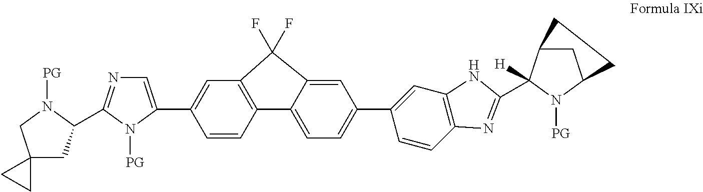
(ii) optional deprotecting the compound of Formula IXi when PG represents a protecting group followed by reaction with compound of Formula 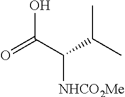
or its reactive derivative to give Ledipasvir of Formula I.

12. (canceled)

13. The process for the preparation of Ledipasvir of Formula I or a pharmaceutically acceptable salt or solvate thereof according to according to claim 2 which comprises:
(i) reacting compound of Formula VII 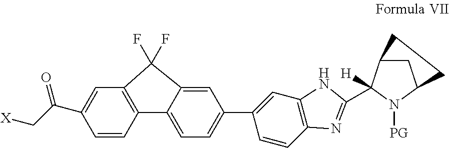
wherein X is halo or leaving group, PG represents protecting group or the group of Formula 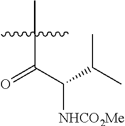
with potassium salt of compound of Formula VIh in acetone 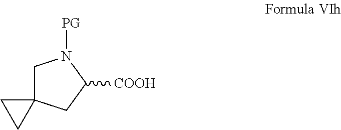
to give compound of Formula IX, 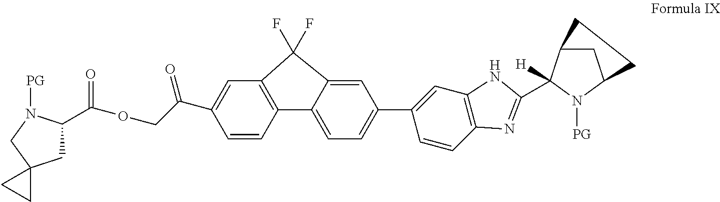
(ii) converting compound of formula IX to Ledipasvir of Formula I.

14. (canceled)

15. (canceled)

16. (canceled)

17. (canceled)

18. Compound of formula IX or its salts, an intermediate of Ledipasvir. 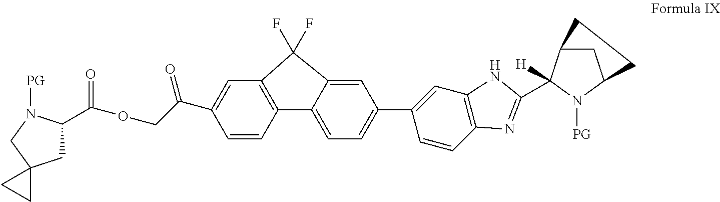
wherein PG represents protecting group.

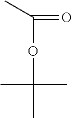

19-34. (canceled)
